(19) United States
(12) Patent Application Publication
(10) Pub. No.: US 2008/0220044 A1
(43) Pub. Date: Sep. 11, 2008

(54) CANCELLOUS CONSTRUCT WITH SUPPORT RING FOR REPAIR OF OSTEOCHONDRAL DEFECTS

(76) Inventors: Eric J. Semler, Piscataway, NJ (US); Roman Shikhanovich, Edison, NJ (US); Alex B. Callahan, Perth Amboy, NJ (US); Katherine G. Truncale, Hillsborough, NJ (US); Judith I. Yannariello-Brown, Somerset, NJ (US)

Correspondence Address: GREENBERG TRAURIG, LLP, 200 PARK AVE., P.O. BOX 677, FLORHAM PARK, NJ, 07932, US

(21) Appl. No.: 12/043,001

(22) Filed: Mar. 5, 2008

Related U.S. Application Data
(60) Provisional application No. 60/904,809, filed on Mar. 6, 2007.

(51) Int. Cl. A61F 2/00; A61K 35/12; A61P 19/00

(52) U.S. Cl. 424/423; 424/549

(57) ABSTRACT

The invention is directed toward an osteochondral repair assembly comprising a shaped allograft construct comprising an unbalanced barbell-shaped cylindrical cancellous bone primary member formed with a mineralized cylindrical base section having a smaller diameter cylindrical stem leading to a second cylindrical section which is demineralized. A mineralized ring-shaped support member is forced over the compressed demineralized second demineralized the aperture of the ring-shaped member to fit around the stem with one ring surface being adjacent the bottom surface to the second cylindrical section and the opposite ring surface being adjacent the upper surface of the mineralized cylindrical base section.

### RELATED APPLICATIONS

This application claims the priority of U.S. Provisional Patent Application No. 60/904,809 filed Mar. 6, 2007, the disclosure of which is incorporated herein by reference in its entirety for all purposes.

### STATEMENT REGARDING FEDERALLY SPONSORED RESEARCH OR DEVELOPMENT

Not applicable.

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention is generally directed toward an allograft implant construct for osteochondral defect repair and is more-specifically directed toward a two piece allograft cancellous bone implant having a cancellous bone base member with a mineralized base section, stem and demineralized top section and a ring-shaped support member which is pulled over the compressed demineralized cancellous top section around the stem. The construct is shaped for an interference fit implantation in a shoulder, knee, hip, or ankle joint, and the construct optionally further contains one or more growth factors impregnated within the construct.

2. Description of the Prior Art

Articular cartilage injury and degeneration present medical problems to the general population which are constantly addressed by orthopedic surgeons. Every year in the United States, over 500,000 arthroplastic or joint repair procedures are performed. These include approximately 125,000 total hip and 150,000 total knee arthroplasties and over 41,000 open arthroscopic procedures to repair cartilaginous defects of the knee.

In the knee joint, the articular cartilage tissue forms a lining which faces the joint cavity on one side and is linked to the subchondral bone plate by a narrow layer of calcified cartilage tissue on the other. Articular cartilage (hyaline cartilage) consists primarily of extracellular matrix with a sparse population of chondrocytes distributed throughput the tissue. Articular cartilage is composed of chondrocytes, type II collagen fibril meshwork, proteoglycans, and water. Active chondrocytes are unique in that they have a relatively low turnover rate and are sparsely distributed within the surrounding matrix. The collagens give the tissue its form and tensile strength and the interaction of proteoglycans with water gives the tissue its stiffness to compression, resilience and durability. The hyaline cartilage provides a low friction bearing surface over the bony parts of the joint. If the lining becomes worn or damaged resulting in lesions, joint movement may be painful or severely restricted. Whereas damaged bone typically can regenerate successfully, hyaline cartilage regeneration is quite limited because of its limited regenerative and reparative abilities.

Articular cartilage lesions generally do not heal, or heal only partially under certain biological conditions due to the lack of nerves, blood vessels and a lymphatic system. The limited reparative capabilities of hyaline cartilage usually results in the generation of repair tissue that lacks the structure and biomechanical properties of normal cartilage. Generally, the healing of the defect results in a fibrocartilaginous repair tissue that lacks the structure and biomedical properties of hyaline cartilage and degrades over the course of time. Articular cartilage lesions are frequently associated with disability and with symptoms such as joint pain, locking phenomena and reduced or disturbed function. These lesions, are difficult to treat because of the distinctive structure and function of hyaline cartilage. Such lesions are believed to progress to severe forms of osteoarthritis. Osteoarthritis is the leading cause of disability and impairment in middle-aged and older individuals, entailing significant economic, social arid psychological costs. Each year, osteoarthritis accounts for as many as 39 million physician visits and more than 500,000 hospitalizations. By the year 2020, arthritis is expected to affect almost 60 million persons in the United States and to limit the activity of 11.6 million persons.

There are many current therapeutic methods being used. None of these therapies has resulted in the successful regeneration of hyaline-like tissue that withstands normal joint loading and activity over prolonged periods. Currently, the techniques most widely utilized clinically for cartilage defects and degeneration are not articular cartilage substitution procedures, but rather lavage, arthroscopic debridement, and repair stimulation. The direct transplantation of cells or tissue into a defect and the replacement of the defect with biologic or synthetic substitutions presently accounts for only a small percentage of surgical interventions. The optimum surgical goal is to replace the defects with cartilage-like substitutes so as to provide pain relief, reduce effusions and inflammation, restore function, reduce disability and postpone or alleviate the need for prosthetic replacement.

Lavage and arthroscopic debridement involve irrigation of the joint with solutions of sodium chloride, Ringer or Ringer and lactate. The temporary pain relief is believed to result from removing degenerative cartilage debris, proteolytic enzymes and inflammatory mediators. These techniques provide temporary pain relief, but have little or no potential for further healing.

Repair stimulation is conducted by means of drilling, abrasion arthroplasty or microfracture. Penetration into the subchondral bone induces bleeding and fibrin clot formation which promotes initial repair, however, the tissue formed is fibrous in nature and hot durable. Pain relief is temporary as the tissue exhibits degeneration, loss of resilience, stiffness and wear characteristics overtime.

The periosteum and perichondrium have been shown to contain mesenchymal progenitor cells capable of differentiation and proliferation. They have been used as grafts in both animal and human models to repair articular defects. Few patients over 40 years of age obtain good clinical results, which most likely reflect the decreasing population of osteochondral progenitor cells with increasing age. There have also been problems with adhesion and stability of the grafts, which result in their displacement or loss from the repair site.

Transplantation of cells grown in culture provides another method of introducing a new cell population into chondral and osteochondral defects. CARTICEL7 is a commercial process to culture a patient=s own cartilage cells for use in the repair of cartilage defects in the femoral condyle marketed by Genzyme Biosurgery in the United States and Europe. The procedure uses arthroscopy to take a biopsy from a healthy, less loaded area of articular cartilage. Enzymatic digestion of the harvested tissue releases the cells that are sent to a laboratory where they are grown for a period ranging from 2-5 weeks. Once cultivated, the cells are injected during a more open and extensive knee procedure into areas of defective cartilage where it is hoped that they will facilitate the repair of damaged tissue. An autologous periosteal flap with a cambium layer is used to seal the transplanted cells in place and act as a mechanical barrier. Fibrin glue is used to seal the edges of the flap. This technique preserves the subchondral bone plate and has reported a high success rate. Proponents of this procedure report that it produces satisfactory results, including the ability to return to demanding physical activities, in more than 90% of patients and those biopsy specimens of the tissue in the graft sites show hyaline-like cartilage repair. More work is needed to assess the function and durability of the new tissue arid determine whether it improves joint function and delays or prevents joint degeneration. As with the perichondrial graft, patient/donor age may compromise the success of this procedure as chondrocyte population decreases with increasing age. Disadvantages to this procedure include the need for two separate surgical procedures, potential damage to surrounding cartilage when the periosteal patch is sutured in place, the requirement of demanding microsurgical techniques, and the expensive cost of the procedure resulting from the cell cultivation which is currently not covered by insurance.

Osteochondral transplantation or mosaicplasty involves excising all injured or unstable tissue from the articular defect and creating cylindrical holes in the base of the defect and underlying bone. These holes are filled with autologous cylindrical plugs of healthy cartilage and bone in a mosaic fashion. The filler osteochondral plugs are harvested from a lower weight-bearing area of lesser importance in the same joint. This technique, shown in Prior Art FIG. 2, can be performed as arthroscopic or open procedures. Reports of results of osteochondral plug autografts a small numbers of patients indicate that they decrease pain and improve joint function, however, long-term results have not been reported. Factors that can compromise the results include donor site morbidity, effects of joint incongruity on the opposing surface of the donor site, damage to the chondrocytes at the articular margins of the donor and recipient sites during preparation and implantation, and collapse or settling of the graft over time. The limited availability of sites for harvest of osteochondral autografts restricts the use of this approach to treatment of relatively small articular defects and the healing of the chondral portion of the autograft to the adjacent articular cartilage remains a concern.

Transplantation of large allografts of bone and overlying articular cartilage is another treatment option that involves a greater area than is suitable for autologous cylindrical plugs, as well as for a non-contained defect. The advantages of osteochondral allografts are the potential to restore the anatomic contour of the joint, lack of morbidity related to graft harvesting, greater availability than autografts and the ability to prepare allografts in any size to reconstruct large defects. Clinical experience with fresh and frozen osteochondral allografts shows that these grafts can decrease joint pain, and that the osseous portion of an allograft can heal to the host bone and the chondral portion can function as an articular surface. Drawbacks associated with this methodology in the clinical situation include the scarcity of fresh donor material and problems connected with the handling and storage of frozen tissue. Fresh allografts carry the risk of immune response or disease transmission. Musculoskeletal Transplant Foundation (MTF) has preserved fresh allografts in a media that maintains a cell viability of 50% for 35 days for use as implants. Frozen allografts lack cell viability and have shown a decreased amount of proteoglycan content which contribute to deterioration of the tissue.

A number of United States Patents have been specifically directed towards bone plugs which are implanted into a bone defect. Examples of such bone plugs are U.S. Pat. No. 4,950,296 issued Aug. 21, 1990 which discloses a bone graft device comprising a cortical shell having a selected outer shape and a cavity formed therein for receiving a cancellous plug, which is fitted into the cavity in a manner to expose at least one surface; U.S. Pat. No. 6,039,762 issued Mar. 21, 2000 discloses a cylindrical shell with ah interior body of deactivated bone material and U.S. Pat. No. 6,398,811 issued Jun. 4, 2002 directed toward a bone spacer which has a cylindrical cortical bone plug with an internal through going bore designed to hold a reinforcing member. U.S. Pat. No. 6,383,211 issued May 7, 2002 discloses an invertebral implant having a substantially cylindrical body with a through going bore dimensioned to receive bone growth materials.

U.S. Pat. No. 6,379,385 issued Apr. 30, 2002 discloses an implant base body of spongious bone material into which a load carrying support element is embedded. The support element can take the shape of a diagonal cross or a plurality of cylindrical pins. See also, U.S. Pat. No. 6,294,187 issued Sep. 25, 2001 which is directed to a load bearing osteoimplant made of compressed bone particles in the form of a cylinder. The cylinder is provided with a plurality of through going bores to promote blood flow through the osteoimplant or to hold a demineralized bone and glycerol paste mixture. U.S. Pat. No. 6,096,081 issued Aug. 1, 2000 shows a bone dowel with a cortical end cap or caps at both ends, a brittle cancellous body and a through going bore.

The use of implants for cartilage defects is much more limited. Aside from the fresh allograft implants and autologous implants, U.S. Pat. No. 6,110,209 issued Nov. 5, 1998 shows the use of an autologous articular cartilage cancellous bone paste to fill arthritic defects. The surgical technique is arthroscopic and includes debriding (shaving away loose or fragmented articular cartilage), followed by morselizing the base of the arthritic defect with an awl until bleeding occurs. An osteochondral graft is then harvested from the inner rim of the intercondylar notch using a trephine. The graft is then morselized in a bone graft crusher, mixing the articular cartilage with the cancellous bone. The paste is then pushed into the defect and secured by the adhesive properties of the bleeding bone. The paste can also be mixed with a cartilage growth factor, a plurality of cells, or a biological glue. All patients are kept non-weight bearing for four weeks and used a continuous passive motion machine for six hours each night. Histologic appearance of the biopsies has mainly shown a mixture of fibrocartilage with hyaline cartilage. Concerns associated with this method are harvest site morbidity and availability, similar to the mosaicplasty method.

U.S. Pat. No. 6,379,367 issued Apr. 30, 2002 discloses a plug with a base membrane, a control plug, and a top membrane which overlies the surface of the cartilage covering the defective area of the joint.

SUMMARY OF THE INVENTION

In one embodiment, an osteochondral repair allograft construct implant is formed as an unbalanced barbell-shaped cylindrical cancellous bone base member having a mineralized cylindrical base section and a smaller diameter cylindrical stem extending there from leading to a second cylindrical section which is demineralized. In another embodiment a ring shaped support member is forced over the compressed demineralized second cylindrical section and the aperture of the ring member fits around the stem with a top surface being adjacent the bottom surface of the demineralized cylindrical section and bottom surface being adjacent the upper surface of the mineralized cylindrical base section. In another embodiment, the allograft construct implant is used to repair osteochondral defects and is placed in a bore which has been cut into the patient to remove the lesion defect area. In another embodiment, each osteochondral repair allograft construct implant can support the addition of a variety of growth factors. In another embodiment, the allograft construct implant can support the addition of a variety of chondrogenic (in any portion of the construct) and/or osteogenic (in any portion of the construct save the demineralized top section) growth factors including, but not limited to morselized allogeneic cartilage, growth factors and variants thereof (FGF-2, FGF-5, FGF-7, FGF-9, FGF-11, FGF-21, IGF-1, TGF-β, TGF-β1, BMP-2, BMP-7, PDGF, VEGF), human allogenic or autologous chondrocytes, human allogenic or autologous bone marrow cells, stem cells, demineralized bone matrix, insulin, insulin-like growth factor-1, transforming growth factor-B, interleukin-1 receptor antagonist, hepatocyte growth factor, platelet-derived growth factor, Indian hedgehog and parathyroid hormone-related peptide or bioactive glue. These chondrogrenic and/or osteogenic growth factors or additives can be added throughout the implant or to specific regions of the implant such as the demineralized top section or the mineralized base portion, depending on whether chondrogenesis (any portion of the implant) or osteogenesis (any portion of the implant save the demineralized top section) is the desired outcome.

In another embodiment, the invention provides an allograft implant for joints which provides pain relief, restores normal function and will postpone or alleviate the need for prosthetic replacement.

In another embodiment, the invention provides an osteochondral repair implant which is easily placed ma defect area by the surgeon using an arthroscopic, minimally invasive technique.

In another embodiment, the invention provides an osteochondral repair implant which has load bearing capabilities.

In another embodiment, the invention provides an osteochondral repair procedure which is applicable for both partial and full thickness cartilage lesions that may or may not be associated with damage to the underlying bone.

In another embodiment, the invention provides an implant capable of facilitating bone healing and/or repair of hyaline cartilage.

In another embodiment, the invention provides a cancellous construct which is simultaneously treated with chondrogenic (in any portion of the implant) and/or osteogenic (in any portion of the implant save the demineralized top section) growth factors.

In another embodiment, the invention provides a cancellous construct which is treated with chondrogenic growth factors in the portion of the construct aimed to repair articular cartilage.

In another embodiment, the invention provides a cancellous construct which is treated with chondrogenic growth factors at any portion of the construct.

In another embodiment, the invention provides a cancellous construct which is treated with osteogenic growth factors in any portion of the construct except for the demineralized top portion of the construct.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 shows the anatomy of a knee joint;

FIG. 2 shows a schematic mosaicplasty as known in the prior art;

FIG. 3 shows an assembled perspective view of the inventive cartilage repair construct;

FIG. 4 shows a perspective view of the base member of the construct with an unbalanced barbell configuration;

FIG. 5 shows a perspective view of the ring shaped support member of the construct;

FIG. 6 is a side elevation view of the assembled construct; and

FIG. 7 shows a cross section view of the construct of FIG. 6 taken along line 7′-7′.

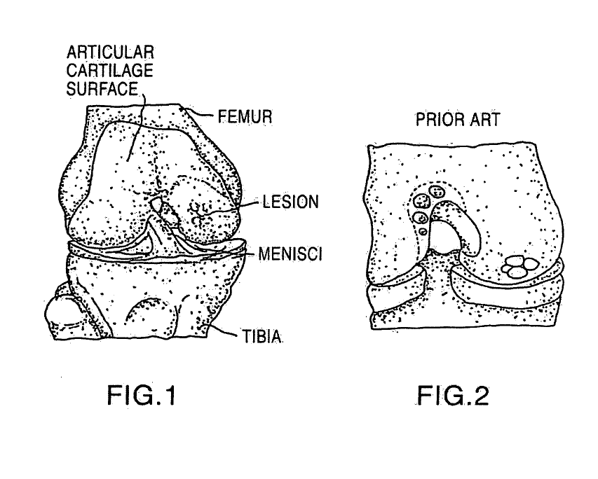

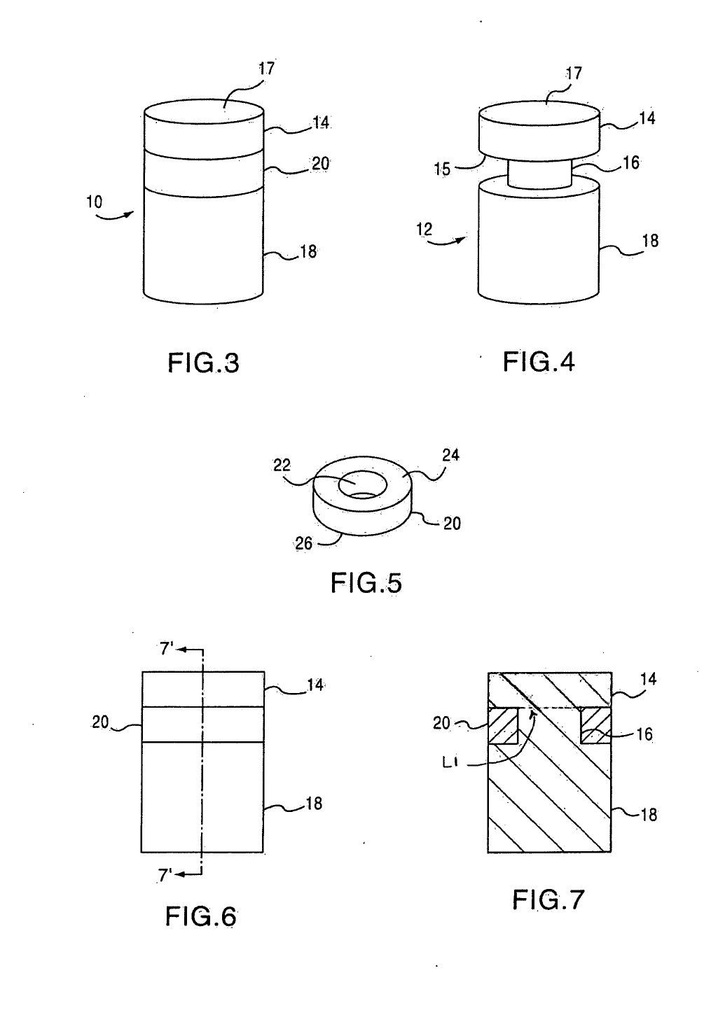

### DESCRIPTION OF THE INVENTION

The term “tissue” is used in the general sense herein to mean any transplantable or implantable tissue, the survivability of which is improved by the methods described herein upon implantation. In particular, the overall durability and longevity of the implant are improved, and host-immune system mediated responses, are substantially eliminated.

The terms “transplant” and “implant” are used interchangeably to refer to tissue, material or cells (xenogeneic or allogeneic) which maybe introduced into the body of a patient.

The terms “autologous” and “autograft” refer to tissue or cells which originate with or are derived from the recipient, whereas the terms “allogeneic” and “allograft” refer to cells and tissue which originate with or are derived from a donor of the same species as the recipient.

The terms “xenogeneic” and “xenograft” refer to cells or tissue which originates with or are derived from a species other than that of the recipient.

The term “growth factor” means a naturally occurring or synthetic compound capable of stimulating cellular proliferation and cellular differentiation. Growth factors are important for regulating a variety of cellular processes.

The term “ELISA” or “Enzyme-Linked Immunosorbent Assay” means a biochemical technique used mainly in immunology to detect the presence of an antibody or an antigen in a sample. The ELISA has been used as a diagnostic tool in medicine and plant pathology, as well as a quality control check in various industries. In simple terms, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light is shone upon the sample, any antigen/antibody complexes will fluoresce so that the amount of antigen in me sample cash be measured.

The term “activated” means a compound of interest that is presumed to be capable of physiologic activity given the conservance of that compound's active binding site. “Activated” compounds are generally identified utilizing antibodies directed to the compound's intact active binding site.

The term “bioburden” means the number of microorganisms with which an object is contaminated.

### Construct

The present invention is directed towards an osteochondral repair construct constructed of cancellous bone taken from allogenic or xenogenic bone sources.

The construct is preferably derived from dense allograft cancellous bone that may originate from proximal or distal femur, proximal or distal tibia, proximal humerus, talus, calcaneus, patella, or ilium. Cancellous tissue is first processed into blocks and then milled into the desired shapes such as a cylinder for this present invention. In a preferred embodiment, a barbell-shaped assembly 10 is milled using a lathe on a cancellous bone cylinder to form an unbalanced primary base member 12 with a top section 14, a cylindrical stem section 16 and a cylindrical base section 18. The top section 14 is milled to have a thickness similar to the thickness of human articular cartilage (e.g., 1.5-3.5 mm) and the diameter of the implant may vary between 5-25 mm. The stem section 16 has a diameter approximately half of the diameter of the entire assembly. The base section 18 has a thickness or length which is preferably larger than the thickness or length of the top section 14 with a ratio preferably ranging from of at least about 1.5 to 1 to about 6:1. During tissue processing, the top section 14 is substantially demineralized by immersing it in dilute acid while the base section 18 remains mineralized.

A mineralized cancellous bone ring shaped secondary member 20 has an aperture 22 with a diameter equal to or slightly greater than the diameter of the stem 16 and an outer diameter which is the same as the diameter of the top section 14 and base section 18. However, if desired, the aperture 22 can be 10% to 40% larger than the diameter of the stem 16. The top surface 24 and bottom surface 26 of the ring shaped member 20 are preferably planar and after assembly the bottom surface 26 is adjacent the top surface 19 of the base section 18 and the top surface 24 is adjacent the bottom surface 15 of the top section 14. While the ring member 20 is preferably constructed of mineralized allograft cancellous bone, it can be constructed of allograft cortical bone or xenograft bone as long as the same have been decellularized. Alternately, the ring member 20 may be constructed of ceramics or biocompatible polymers.

### Demineralization

The top section 14 is substantially demineralized in dilute acid up to a predetermined level (as indicated by broken-line representation L1 in FIG. 7) until the bone contains less than 0.5% wt/wt residual calcium. Subsequently, the resultant tissue form is predominantly Type I collagen, which is sponge-like in nature with an elastic quality. Following decalcification, the tissue is further cleaned, brought to a physiological pH level of about 7 and may also be treated so that the cancellous tissue is non-osteoinductive. This inactivation of inherent ostebinductivity may be accomplished via chemical or thermal treatment or by high energy irradiation. The cancellous top section 14 is preferably treated with an oxidizing agent such as hydrogen peroxide in order to render it non-osteoinductive.

Following demineralization the top section 14 is spongy and deformable allowing it to be squeezed through the center aperture 22 of the ring member 20. After the implant has been assembled, morselized cartilage particles combined with a carrier or growth factor may be added to the top section 14. If desired, the open cancellous structure of the top section 14 may be loaded with a cartilage paste or gel as noted below and/or one or more additives namely recombinant or native or variant growth factors (FGF-2, FGF-5, FGF-7, FGF-9, FGF-11, FGF-21, IGF-1, TGF-β, BMP-2, BMP-4, BMP-7, PDGF, VEGF), human allogenic or autologous chondrocytes, human allogenic cells, human allogenic or autologous bone marrow cells, human allogenic or autologous stem cells, demineralized bone matrix, insulin, insulin-like growth factor-1, interleukin-1 receptor antagonist, hepatocyte growth factor, platelet-derived growth factor, Indian hedgehog, parathyroid hormone-related peptide, viral vectors for growth factor or DNA delivery, nanoparticles, or platelet-rich plasma. This design enables the fabrication of an implant that possesses a relatively uniform substantially demineralized top section that is distinct from the mineralized base section.

Incorporation of Additives into the Construct

The demineralized portion of the construct can be provided with a matrix of minced cartilage putty or gel consisting of minced or milled allograft cartilage which has been lyophilized so that its water content ranges from 0.1% to 8.0% ranging from 25% to 50% by weight, mixed with a carrier of sodium hyaluronate solution (HA) (molecular weight ranging from 7.0×10⁵ to 1.2×10⁶) or any other bioabsorbable carrier such as hyaluronic acid and its derivatives, gelatin, collagen, chitosan, alginate, buffered PBS, Dextran CMC, or other polymers, the carrier ranging from ranging from 75% to 25% by weight. In one embodiment, the cartilage is milled or morselized to a size less than or equal to 212 μm. In another embodiment, the cartilage is milled or morselized to a size of from about 5 μm to about 212 μm. In another embodiment, the cartilage is milled or morselized to a size of from about 6 μm to about 10 μm. In another embodiment, the cartilage can be milled or morselized to a size of less than or equal to about 5 μm. The small size of the particulate cartilage facilitates can increase exposure or release of various growth factors due to the increased aggregate surface area of the particulate cartilage used.

The cartilage particles can contain endogenous growth factors. These endogenous growth factors can he extracted from the cartilage particles by the method outlined in Example 1 and detected by the method outlined in Example 3. The levels of these growth factors may be similar to or greater than the levels of endogenous growth factors in intact cartilage. The endogenous growth factors from intact cartilage can be extracted by the method outlined in Example 2 and detected using the method outlined in Example 3. Exogenous growth factors can also be combined with the cartilage particulate. In one embodiment, cartilage is recovered from deceased human donors, and the tissue is treated with a soft tissue processing system for bioburden reduction, for example, of the type as disclosed in U.S. patent application Ser. No. 11/375,026 (U.S. Publication No. 2006/0275377) filed Mar. 15, 2006, the entire disclosure of which is incorporated herein by reference in its entirety for all purposes The cartilage is then lyophilized, milled, then sieved to yield particle sizes of, on average, less than or equal to 212 microns. The cartilage particles are mixed with a growth factor in an aqueous vehicle, then the particles can either be lypohilized and stored dry at room temperature or frozen, or used immediately. For example, particles containing chondrogenic growth factor can be added to any portion of the allograft construct, and particles containing osteogenic growth factor can be added to any portion of the allograft construct save the demineralized cancellous cap. The mixture containing the cartilage particles arid growth factor can be lyophilized for storage.

The growth factor can be any one of a variety of growth factors known to promote wound healing, cartilage and/or bone development (e.g. BMP's partictularly BMP-2, FGF's particularly FGF-2 and -9 and a variant of FGF-2 [ProChon Biotech, Ltd of Israel], IGF, VEGF, PDGF, etc.), the vehicle used to solubilize the growth factor arid adsorb it into the cartilage particles can be saline, water, PBS, Ringers, etc.

In one embodiment, the resulting enhanced cartilage particles can contain levels of growth factors that are greater than that found in intact cartilage. In another embodiment, the cartilage particle mixture can be infused into all or part of the construct. If desired, the cartilage particle mixture can be infused primarily into the demineralized end of the primary member of the construct.

It is further envisioned that cells which have been collected from the patient or grown outside the patient can be inserted into the entire construct or into the cancellous demineralized top section 14 matrix before, during or after deposit of the construct 10 into the defect area. Such cells include, for example, allogenic or autologous bone marrow cells, stem cells and chondrocyte cells. The cellular density pf the cells preferably ranges from 1.0×10⁸ to 5.0×10⁸ or from about 100 million to about 500 million cells per cc of putty or gel mixture.

### Placement of Construct

The construct 10 is placed in an osteochondral defect area bore which has been cut in the lesion area of a patient with the upper surface 17 of the top section 14 being slightly proud (i.e., above), slightly below, or substantially flush with the surface of the original cartilage surrounding the defect area remaining at the site being treated. The construct 10 has a length which can be the same as the depth of the defect or more or less than the depth of the bore. If the construct 10 is the same as the depth of the bore, the base of the implant is supported by the bottom surface of the bore and the top surface 17 is substantially level with the articular cartilage. If the construct 10 is of a lesser length, the base of the construct is not supported but support is provided by the wall of the defect area bore of respective cut out area as the plug is interference fit within the bore or cut out area with the cap being slightly proud, slightly below, or flush with the surrounding articular cartilage depending on the surgeon's preference. With such load bearing support, the graft surface is not damaged by weight or bearing loads which can cause micromotion interfering with the graft interface producing fibrous tissue interfaces and subchondral cysts.

In operation, the lesion or defect is removed by cutting a blind bore removing a lesion in the implant area. The construct 10 is then placed in the bore or cut away area in an interface fit with the surrounding walls.

If the construct is moveable within the bore, suitable organic glue material can be used to keep the implant fixed in place in the implant area. Suitable organic glue material can also be used to keep the additives in the construct within the construct following implantation into the defect site. Suitable organic glue material can be found commercially, such as for example: TISSEEL7 or TISSUCOL 7 (fibrin based adhesive, Immuno AG, Austria), Adhesive Protein (Sigma Chemical, USA), Dow Corning Medical Adhesive B (Dow Corning, USA), fibrinogen thrombin, elastin, collagen, casein, albumin, keratin and the like.

### EXAMPLES

### Example 1

### Processed Cartilage Particle Extraction

Cartilage is recovered from deceased human donors, and the tissue is treated with a soft tissue processing system for bioburden reduction. The cartilage is then lyophilized, milled, then sieved to yield particle sizes of, on average, less than or equal to 212 microns. The cartilage particles are again lyophilized prior to storage or extraction. The particles are extracted in guanidine HCl by incubating at 4° C. on an orbital shaker at 60 rpm for 24 hr, followed by dialysis (8k MWCO membrane dialysis tube) in 0.05M Tris HCl for 15 hrs at 4° C. The dialysis solution was then replaced and the dialysis continued for another 8 hrs at 4° C. The post-dialysis extracts were stored at −70° C. until ELISA analysis.

### Example 2

### Native Cartilage Extraction

Cartilage is recovered from deceased human donors. The tissue is lyophilized, then extracted in Guanidine HCl without any further pre-treatments. The cartilage is extracted in guanidine HCl by incubating at 4° C. on an orbital shaker at 60 rpm for 24 hr, followed by dialysis (8k MWCO membrane dialysis tube) in 0.05M TrisHCl for 15 hrs at 4° C. The dialysis solution was then replaced and the dialysis continued for another 8 hrs at 4° C. The post dialysis extracts were stored at −70° C. until ELISA analysis.

### Example 3

### Quantification Of Endogenous Growth Factors Present In Native And Processed Cartilage

0.25 g of cartilage particles were weighed out for each donor. The cartilage particles were transferred to tubes containing 5 ml of extraction solution (4M Guanidine HCl in TrisHCl). The cartilage particles were incubated at 4° C. on the orbital shaker at 60 rpm for 24 hr, followed by dialysis (8k MWCO membrane dialysis tube) in 0.05M TrisHCl for 15 hrs at 4° C. The dialysis solution was then replaced and the dialysis continued for another 8 hrs at 4° C. The post-dialysis extracts were stored at −70° C. until ELISA run. Notably, the above protocol can also be utilized in order to determine the total growth factor concentration (e.g. exogenous plus endogenous) present in a device of the instant invention.

TABLE 1 demonstrates the relative concentration of endogenous TGF-β1 found in cartilage particles of the present invention derived from various subjects, and from native (e.g., annulled) cartilage from a subject.

TABLE 1
Quantification Of Total Endogenous TGF-β1 Present In Processed
Cartilage Particles And In Native Cartilage

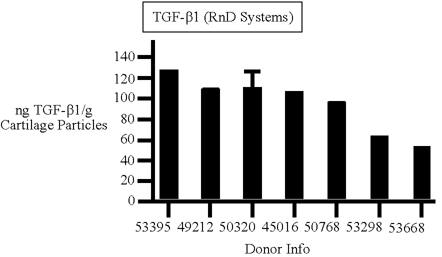

| Donor | | | | |
|---|---|---|---|---|
| Number | Age | Sex | Height | Weight (lb) |
| 53395 | 39 | M | 6′4″ | 295 |
| 49212 | 44 | M | 6′0″ | 244 |
| 50320 | 20 | M | 6′2″ | 231 |
| 45016 | 23 | M | 5′10″ | 180 |
| 50768 | 26 | M | 5′6″ | 163 |
| 53298 | 25 | M | 5′1O″ | 174 |
| 53668 | 37 | M | 6′0″ | 273 |

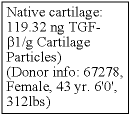

TABLE 2 demonstrates the relative concentration of endogenous bioactive TGF-β1 found in cartilage particles manufactured according to Example 1 of the present invention and derived from various subjects.

TABLE 2
Quantification Of Bioactive Endogenous TGF-β1 Present In Processed
Cartilage Particles

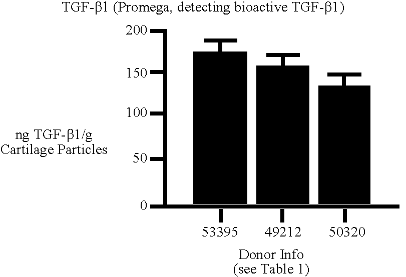

TABLE 3 demonstrates the relative concentration of endogenous FGF-2 found in cartilage particles of the present invention manufactured in accordance with Example 1 of the present invention and derived from various subjects.

TABLE 3
Quantification Of Total Endogenous FGF-2 Present In Processed Cartilage
Particles

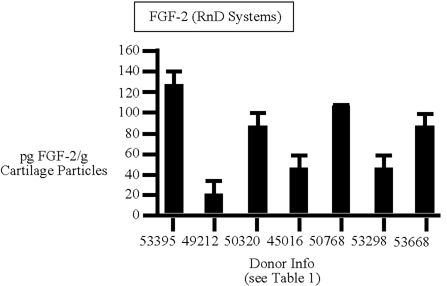

TABLE 4 demonstrates the relative concentration of endogenous BMP-2 found in cartilage particles of the present invention manufactured in accordance with Example 1 of the present invention and derived from various subjects.

TABLE 4
Quantification Of Total Endogenous BMP-2 Present In Processed
Cartilage Particles And In Native Cartilage

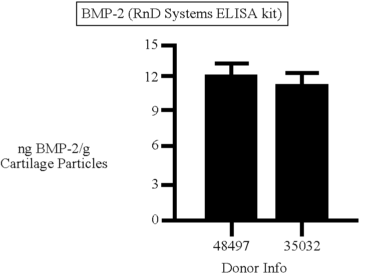

Donor info:
48497 (42 yr, Male, 6′0″, 240 lbs)
35032 (46yr, Male, 5′11″, 305 lbs)

The results shown above in Tables 1-4 indicate that both processed cartilage particles prepared in accordance with the method of Example 1, and native cartilage prepared in accordance with the method of Example 2 retains a concentration of endogenous TGF-β1. The results shown above in Tables 1-4 further indicate that processed cartilage particles prepared in accordance with the method of Example 1 also retain a concentration of active endogenous TGF-β1; of endogenous BMP-2; and of endogenous FGF-2.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims.

What we claim is:

1. A construct for repairing an osteochondral defect, comprising a base member derived from bone, said base member having a first section, which is mineralized, and a second section, which has a substantially demineralized region, said first and second sections being connected by a stem section; and a ring shaped member mounted around said stem section of said base member.

2. An osteochondral repair construct as recited in claim 1, wherein each of said first and second sections has a cylindrical shape; and wherein said ring-shaped member has a cylindrical shape.

3. An osteochondral repair construct as recited in claim 2, wherein said first and second sections and said stem section are formed integrally with each other, whereby said base member has a monolithic construction.

4. An osteochondral repair construct as recited in claim 3, wherein each of said first and second sections has a first diameter, and wherein said stem section has a second diameter, which is smaller than said first diameter.

5. An osteochondral repair construct as recited in claim 4, wherein said first and second sections are located at opposite ends of said base member; and wherein said stem section is located intermediate said opposite ends of said base member, whereby said base member has a barbell-like shape.

6. An osteochondral repair construct as recited in claim 5, wherein said first section has a first thickness; and wherein said second section has a second thickness, which is less than said first thickness.

7. An osteochondral repair construct as recited in claim 1, wherein said second section of said base member is demineralized so as to have a residual calcium content not greater than about 0.5% wt/wt.

8. An osteochondral repair construct as recited in claim 1, wherein said stem section of said base member is at least partially demineralized.

9. An osteochondral repair construct as recited in claim 1, wherein said ring-shaped member is formed from allograft cancellous or cortical bone.

10. An osteochondral repair construct as recited in claim 1, wherein said ring-shaped member is formed from xenograft cancellous or cortical bone.

11. An osteochondral repair construct as recited in claim 1, wherein said demineralized region of said second section of said base member is treated to be non-osteoinductive.

12. An osteochondral repair construct as recited in claim 1, wherein said base member is formed from allograft cancellous bone.

13. An osteochondral repair construct as recited in claim 1, wherein said base member is formed from xenograft cancellous bone.

14. An osteochondral repair construct as recited in claim 1, wherein said stem section has a first diameter; and wherein said ring member includes an aperture having a second diameter, which is similar to said first diameter of said stem section.

15. An osteochondral repair construct as recited in claim 14, wherein said second diameter of said ring-shaped member is larger than said first diameter of said stem section by about 10% to about 40%.

16. An osteochondral repair construct as recited in claim 1, wherein said ring-shaped member is constructed of materials taken from a group consisting of allograft bone, xenograft bone, ceramics and biocompatible plastic polymers.

17. An osteochondral repair construct as recited in claim 1, wherein said second section of said base member contains cartilage particles.

18. An osteochondral repair construct as recited in claim 1, wherein said second section of said base member contains cartilage particles mixed in a biocompatible carrier.

19. An osteochondral repair construct as recited in claim 18, wherein said cartilage particles are derived from allograft or autograft cartilage.

20. An osteochondral repair construct as recited in claim 18, wherein said cartilage particles contain endogenous growth factors.

21. An osteochondral repair construct as recited in claim 18, wherein said cartilage particles contain at least one additive taken from a group consisting of and variants thereof (FGF-2, FGF-5, FGF-7, FGF-9, FGF-11, FGF-21, IGF-1, TGF-b, TGF-B1, BMP-2, BMP-4, BMP-7, PDGF, VEGF), human allogeneic or autologous chondrocytes, human allogeneic or autologous bone marrow cells, stem cells, insulin, insulin-like growth factor-1, transforming growth factor-B, interleukin-1 receptor antagonist, hepatocyte growth factor, platelet-derived growth factor, Indian hedgehog and parathyroid hormone-related peptide, bioactive glue, viral vectors for growth factor or DNA delivery, nanoparticles, or platelet-rich plasma.

22. An osteochondral repair construct as recited in claim 18, wherein said cartilage particles contain at least one additive taken from a group consisting of growth factors and variants of FGF-2, FGF-5, FGF-7, FGF-9, FGF-11 and FGF-21.

23. An osteochondral repair construct as recited in claim 18, wherein said cartilage particles are milled.

24. An osteochondral repair construct as recited in claim 18, wherein said cartilage particles are morselized.

25. An osteochondral construct as recited in claim 1, wherein at least one of said base member and said ring-shaped member contains at least one additive taken from a group consisting of growth factors and variants thereof (FGF-2, FGF-5, FGF-7, FGF-9, FGF-11, FGF-21, IGF-1, TGF-b, TGF-B1, BMP-2, BMP-4, BMP-7, PDGF, VEGF), human allogeneic or autologous chondrocytes, human allogeneic or autologous bone marrow cells, stem cells, demineralized bone matrix, allograft or autograft cartilage particles, insulin, insulin-like growth factor-1, transforming growth factor-B, interleukin-1 receptor antagonist, hepatocyte growth factor, platelets-derived growth factor, Indian hedgehog and parathyroid hormone-related peptide, bioactive glue, viral vectors for growth factor or DNA delivery, nanoparticles, or platelet-rich plasma.

26. An osteochondral construct as recited in claim 1, wherein said second section of said base member contains at least one additive taken from a group consisting of growth factors and variants thereof (FGF-2, FGF-5, FGF-7, FGF-9, FGF-11, FGF-21, IGF-1, TGF-b, TGF-B1, BMP-2, BMP-4, BMP-7, PDGF, VEGF), human allogeneic or autologous chondrocytes, human allogeneic or autologous bone marrow cells, stem cells, demineralized bone matrix, allograft or autograft cartilage particles, insulin, insulin-like growth factor-1, transforming growth factor-B, interleukin-1 receptor antagonist, hepatocyte growth factor, platelet-derived growth factor, Indian hedgehog and parathyroid hormone-related peptide, bioactive glue, viral vectors for growth factor or DNA delivery, nanoparticles, or platelet-rich plasma.

27. An osteochondral construct as recited in claim 1, wherein said second section of said base member contains at least one chondrogenic additive and said first section of said base member contains at least one osteogenic additive.

28. An osteochondral construct as recited in claim 1, wherein said second section of said base member contains at least one chondrogenic additive; wherein said first section of said base member contains at least one osteogenic additive; and wherein said ring-shaped member contains at least one osteogenic additive.

29. An osteochondral repair construct as recited in claim 1, wherein said second section of said base member contains at least one additive taken from a group consisting of growth factors and variants of FGF-2, FGF-5, FGF-7, FGF-9, FGF-11 and FGF-21.

30. A process for assembling an osteochondral repair construct having a base member, which includes a stem section intermediate opposite ends of said base member, and a ring-shaped member, which is mountable on said stem section, said method comprising the steps of:
a. demineralizing one of said ends of said base member such that said one end is compressible;
b. compressing said one end of said base member;
c. inserting said ring-shaped member over said one end of said base member while said one end is compressed; and
d. locating said ring-shaped member around said stem section of said base member.

31. The process recited in claim 1, further comprising the steps of
e. providing cartilage particles mixed in a biocompatible carrier to form a cartilage particle mixture; and
f. incorporating said cartilage particle mixture into said one end of said base member.
